(19) United States
(12) Patent Application Publication
(10) Pub. No.: US 2003/0021844 A1
(43) Pub. Date: Jan. 30, 2003

(54) Immediate-release oral pellet comprising polyglycolysed glycerides, and manufacturing process

(76) Inventors: Philippe Barthelemy, Myons (FR); Nabil Farah, Lyon (FR); Pascale Roussin, Lyon (FR)

Correspondence Address: NORRIS MCLAUGHLIN & MARCUS, P.A., P O BOX 1018, SOMERVILLE, NJ, 08876

(21) Appl. No.: 10/198,273

(22) Filed: Jul. 17, 2002

Related U.S. Application Data
(62) Division of application No. 09/622,704, filed on Aug. 21, 2000.
Application No. 09/622,704 is a 371 of international application No. PCT/FR99/00406, filed on Feb. 24, 1999.

(30) Foreign Application Priority Data
Mar. 4, 1998 (FR) FR 98/02855
Feb. 24, 1999 (WO) WO 99/44589

(51) Int. Cl. A61K 9/00; A61K 9/22

(52) U.S. Cl. 424/468

(57) ABSTRACT

The invention concerns a pellet for immediate release of an active substance to be ingested, said pellet comprising at least one active substance, a binding agent and a diluting agent, characterised in that said binding agent: comprises a mixture of glycerides subjected to polyglycolysis whereof the fatty acids comprise at least 8 carbon atoms; has a melting point not less than 37° C.; has a hydrophilic/lipophilic balance not less than 10, said binding agent being capable of enhancing the active substance bioavailability.

The invention relates to a specific pharmaceutical dosage form known as a “pellet” for the immediate release of active substance which is intended to be ingested and is capable of improving the bioavailability of the active substance. It also relates to the process for the manufacture of this pellet.

The Applicant Company has developed compositions which can be administered orally which are capable of improving the bioavailability of the active principle. These compositions are known under the term SMEDDS (Self-Microemulsifying Drug Delivery System).

SMEDDS are disclosed more particularly in the document of the Applicant EP-A-0,670,715. They are composed of a mixture of one or more active principles with a lipophilic phase, a surfactant, a cosurfactant, the characteristics of which are determined so that the final product is capable of forming a microemulsion on contact with a given volume of physiological fluid.

Some products sold by the Applicant, composed of saturated and/or unsaturated fatty acids and of esters of these fatty acids, can be used as the lipophilic phase, surfactant and cosurfactant, such as, for example, the combination respectively of Gelucire® 44/14, Labrafac® CM10 and Lauroglycole which are described in Examples 1 and 2 of the above-mentioned document.

In practice, the process for the manufacture of these compositions consists:

first of all, in liquefying the lipophilic phase, if necessary, by heating (for example Gelucire® 44/14, which is solid at room temperature);

in then mixing the said lipophilic phase with the indicated proportions of surfactant (for example Labrafac® CM10) and of cosurfactant (Lauroglycol®);

and then dispersing, with stirring, the active substance or substances in the mixture obtained.

The mixture obtained is injected directly in the liquid state into hard gelatin capsules or soft capsules, if appropriate before solidification at room temperature.

However, this packaging method has a number of disadvantages, since the filling of the hard gelatin capsules with a formula of SMEDDS type in the liquid state requires specific additional and expensive equipment not always available to manufacturers of finished products. This is because it is necessary to have available a jacketed/stirred storage system and an accurate injection pump system. In addition, the manufacture of capsules also requires specific equipment composed of a machine for bandrolling gelatin capsules, it being possible, furthermore, for this type of packaging to exhibit a risk of leakages of^{—} the SMEDDS from the capsule. It follows that the manufacture of these capsules has to be subcontracted to specialist custom moulders.

In order to increase the bioavailability of the active principle and more particularly that of coenzyme Q₁₀, the document U.S. Pat. No. 4,869,900 has provided a composition comprising, in addition to coenzyme Q₁₀ in a proportion of 2 to 17%, lecithin, used as solubilizing agent, in a proportion of 50 to 70% and Gelucire® 50/13 or 44/14, used solely as surfactant (that is to say, emulsifier), in a proportion of between 20 and 48%. In practice, the coenzyme Q₁₀ is brought into contact with the surfactant at 60° C. and then the product obtained is mixed, with stirring, with the lecithin. The fluid mixture obtained is subsequently adsorbed on an inert material of the colloidal silica type. The resulting granules are then bagged up or subjected to a compression stage in the presence of conventional additives, so as to obtain tablets. In addition, it is observed (Example 8 and column 2, line 50) that the bioavailability of the coenzyme Q₁₀/lecithin mixture and that of the coenzyme Q₁/Gelucire® mixture is very low, whereas that of the coenzyme Q₁₀/lecithin/Gelucire® mixture is increased, which implies that the lecithin/Gelucire® combination exerts a synergistic effect with respect to the improvement in the bioavailability. On the other hand, nothing is indicated regarding the rate of release of the active principle.

The document WO 94/23700 has also provided a solid pharmaceutical composition in the form of pellets for the immediate release of weakly soluble active principle which are manufactured as follows. The active principle is first of all dissolved in a liquid phase composed either of oils, polar solvents, fatty substances or ionic or nonionic surfactants (see page 3, lines 1 to 10). The product obtained is subsequently agglomerated on particles of microcrystalline cellulose by wet granulation requiring water. The agglomerated particles then obtained are subsequently subjected to two additional manufacturing stages, respectively a first stage of extrusion in an extruder and a second stage of spheronization in a spheronizer. In other words, the pellets are obtained by a lengthy multi-stage process requiring the presence of water, which can have a negative effect on the stability of the active principle if the latter is sensitive to oxidation phenomena.

Furthermore, the document WO 93/18753 has disclosed a process for the manufacture of pellets for the prolonged release of active substance by melt pelletization, which consists, in a single stage, in agglomerating, densifying and spheronizing microparticles. In the process disclosed, the binder is composed of a water-insoluble substance of wax type with a melting point of greater than 40° C.

The problem which the invention intends to solve is therefore that of supplying a solid composition, in particular in the form of pellets for the immediate release of active principle, comprising the lowest possible number of constituents while improving the relative bioavailability. The relative bioavailability is the bioavailability obtained by the oral route with respect to the bioavailability obtained by the injectable route.

Another problem of the invention is to supply a formulation of the SMEDDS type which can be packaged other than in the liquid state without requiring the specific and expensive equipment described above.

Another aim of the invention is to provide a composition of the SMEDDS type which can be formulated and packaged not only in the form of hard gelatin capsules but also in other conventional pharmaceutical dosage forms.

As regards the process for the manufacture of the pellets, the problem which the invention intends to solve is that of developing a process requiring a minimum number of stages.

Likewise, and in connection with the product obtained, another problem which the invention intends to solve consists in reducing the number of constituents necessary for the manufacture.

To solve the problem of supplying a solid composition for the immediate release of active principle comprising the lowest possible number of constituents while exhibiting an improved bioavailability, the invention provides a pellet for the immediate release of active substance which is intended to be ingested, the said pellet comprising at least one active substance, one binder and one diluent, characterized in that the binder:

comprises a mixture of polyglycolysed glycerides, the fatty acids of which comprise at least 8 carbon atoms,

exhibits a melting point of greater than or equal to 37° C., p1 exhibits a hydrophilic-lipophilic balance of greater than or equal to 10, the said binder being capable of improving the bioavailability of the active substance.

In the continuation of the description and in the claims, the term “pellets” denotes multiparticulate agglomerated solid forms with a size varying between 0.5 and 5 mm, the surface of which is regarded as spherical and the surface condition of which is regarded as homogeneous.

Likewise, the expression “polyglycolysed glycerides, the fatty acids of which comprise at least 8 carbon atoms” denotes the product of the reaction between a polyethylene glycol with a molecular weight of between 200 and 1500 and a starting oil, the said oil consisting of a mixture of triglycerides with fatty acids chosen from the group comprising caprylic acid, capric acid, myristic acid, palmitic acid, stearic acid, oleic acid and linoleic acid, alone or as a mixture.

In other words, the product resulting from this reaction is a mixture of mono-, di- and triglycerides and of polyethylene glycol (PEG) mono- and diester, the polyethylene glycol having a molecular weight of between 200 and 1500, optionally of glycerol and of free PEG.

In fact, the Applicant has found that the polyglycolysed glycerides, the fatty acids of which comprise at least 8 carbon atoms, selected were not only capable of acting as binder, binding and coating all the constituents, thus ensuring the cohesion of the particles to one another, but also conferred an improved bioavailability on the final pellets.

In addition, the pellets of the invention comprise a minimum of only three constituents (active substance, binder and diluent) in contrast to the prior art, where the presence, on the one hand, of a full-fledged solubilizing agent and, on the other hand, of a solvent of the water type, making it possible to agglomerate the particles by wet granulation, was essential.

The binder advantageously comprises a mixture of saturated polyglycolysed glycerides, the fatty acids of which comprise from 8 to 18 carbon atoms (C₈-C₁₈), exhibits a melting point of 44° C. and exhibits a hydrophilic-lipophilic balance of 14.

The term “C₈-C₁₈” denotes mixtures, in significant and variable proportions, of caprylic (C₈), capric (C₁₀), lauric (C₁₂), myristic (C₁₄), palmitic (C₁₆) and stearic (C₁₈) acids, when these acids are saturated, and the corresponding unsaturated C₈-C₁₈ acids. The proportions of these fatty acids can vary according to the starting oils.

The abovementioned mixture preferably comprises from 15 to 40% by weight of glycerol esters, the remainder to 100% being composed of PEG esters.

According to another embodiment, the binder comprises a mixture of polyglycolysed glycerides, the fatty acids of which comprise 22 carbon atoms (behenic acid), alone or in combination with C₈-C₁₈ fatty acids, in order to adjust the film-forming and binding properties of the mixture and to adjust the release characteristics desired for the active substance employed.

In addition, these pellets exhibit the advantage of being able to be incorporated as such into hard gelatin capsules, in other words without a prior liquefaction stage, thus not requiring specific equipment but simple equipment, which makes it possible to employ multiparticulate solid forms.

According to another characteristic of the invention, the concentration of binder represents between 10 and 40% by weight of the pellet, advantageously from 15 to 20% by weight.

For a concentration of less than 10%, the pellets are observed to lack cohesion and to be very heterogeneous in size.

For a concentration of greater than 40%, the pellets are observed to agglomerate, forming compact and soft lumps.

In order to solve the problem of obtaining a formulation of the SMEDDS® type, the binder can additionally comprise an additive chosen from the group consisting of polyethylene glycol, esters of glycerol, esters of polyethylene glycol, esters of propylene glycol and esters of polyglycerol with saturated and/or unsaturated fatty acids, alone or as a mixture.

This is because the choice of the additive makes it possible to adjust at the same time the melting point and the hydrophilic-lipophilic balance of the binder, and also to improve its affinity with the active substance.

In order to optimize the surface tension between the diluent and the binder, use is made of a soluble or insoluble diluent chosen from excipients which are conventional in pharmaceuticals.

The diluent is advantageously chosen from the group consisting of calcium diphosphate, lactose and other polyols, cellulose esters and silica derivatives.

In order to promote the homogeneity of the mixing of each of the constituents and to make possible the uniform release of the active substance, the diluent is provided in the micronized form with a size of between 1 and 300 micrometers, advantageously between 5 and 60 micrometers.

For a size of less than 1 micrometer, the use of the powder is observed to be problematic because of its lightness and its high bulk volume.

For a size of greater than 300 micrometers, the size of the pellets is observed to be heterogeneous.

Likewise, the active substance is advantageously provided in the micronized form with a size of between 1 and 300 micrometers, advantageously between 5 and 60 micrometers.

As already said, another problem which the invention intends to solve is that of developing a process requiring a minimum number of stages.

To do this, the process for the manufacture of pellets of the invention is characterized in that:

first of all, the active substance, the binder and the diluent are introduced into a mixer equipped with a planetary stirring and heating system,

the constituents are then stirred, while heating, until a temperature close to the melting point of the binder is reached,

the stirring of the mixture obtained is continued until the diluent and the active substance are homogeneously dispersed in the binder,

the stirring and the temperature are maintained in order to make possible the agglomeration, the densification and the spheronization of the particles formed until individual pellets are obtained,

the individual pellets are discharged from the mixer, still with stirring and at a temperature substantially equal to that of the melting point of the binder,

finally, the pellets are recovered and are cooled to a temperature which makes it possible to solidify and to maintain the individual pellets separate from one another.

This is because it has been found that cooling the pellets outside the machine makes it possible to avoid the phenomena of adhesion of the pellets, in contrast to other cooling conditions.

The pellets are advantageously cooled to a temperature of between 4 and 30° C.

According to a first embodiment of the process of the invention, the cooling of the pellets is carried out on trays.

According to a second embodiment of the invention, the cooling of the pellets is carried out by means of a fluidized air bed.

Furthermore, and according to another characteristic, the stirring is between 300 and 1000 revolutions per minute.

For stirring of less than 300 revolutions per minute, no success is achieved in obtaining a homogeneous mixture.

For stirring of greater than 1000 revolutions, excessively high heating of the mixture is observed, preventing the formation of pellets.

Furthermore, the process of the invention makes it possible to prepare varied formulations.

This is because the cooled pellets can be incorporated in hard gelatin capsules.

Likewise, they can be incorporated in sachets.

In other words, the selection of the binder makes it possible, by this preparation process, to employ formulations exhibiting an improved bioavailability not in the liquid form but in the solid form and thus to vary the formulations. Furthermore, and as already said, the formulations can be prepared in a single operation using equipment conventional in the pharmaceutical industry for multiparticulate solid forms.

The invention also relates to the pellets which can be obtained by the process described above.

The invention and the advantages which result therefrom will emerge clearly from the following implementational examples in support of the appended figures, in which:

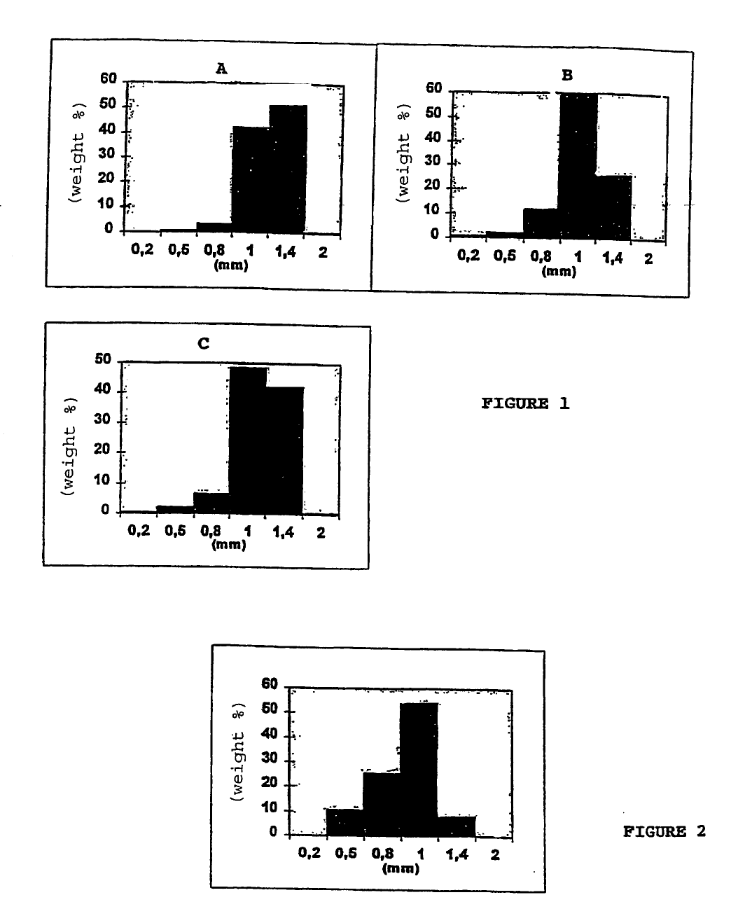

FIG. 1 represents the size distribution for pellets manufactured in accordance with the invention for stirring times of 35 minutes (A), 32 minutes (B) and 29 minutes (C) using Gelucire® 44/14 as binder;

FIG. 2 represents the size distribution of pellets manufactured in accordance with the invention using Gelucire® 50/13 as binder.

In the following examples, the pellets were manufactured in a high-speed mixer of the VG 25 type from Glatt.

### EXAMPLE 1

Each of the constituents, namely respectively:

| | |
|---|---|
| active substance (indomethacin) | 4% |
| in the micronized form: | |
| binder (Gelucire ® 44/14): | 16% |
| diluent (lactose) in the | 80% |
| micronized form: | |

is introduced into the mixer.

Micronization is carried out to a size of approximately 60 micrometers.

The constituents of the mixture are stirred at the rate of 600 revolutions/minute while heating the jacket to a temperature substantially equal to the melting point of the Gelucire, namely to 44° C. The melting temperature is obtained after five minutes.

The stirring time is subsequently varied as follows:

A: 35 minutes

B: 32 minutes

C: 29 minutes

Once the agglomeration, the densification and the spheronization of the particles formed is obtained, the pellets are discharged by means of outlet valves, still with stirring.

In other words, the cooling of the pellets does not take place in the machine, as was the case previously, but outside the machine. This is because it has been found that this technique makes it possible to avoid the adhesion of the pellets to the walls of the machine.

The individual pellets are placed on trays, in order to allow them to be cooled to between 19 and 25° C.

Under these conditions, it is noticed that completely separate pellets are obtained which exhibit a virtually uniform size distribution in accordance with FIG. 1.

The pellets obtained can subsequently be incorporated in hard gelatin capsules or sachets.

They can also be subjected to a compression stage which makes it possible to obtain tablets.

### EXAMPLE 2

The preceding example is repeated using Gelucire® 50/13 of the Applicant as diluent.

Under the same conditions, with a stirring time after melting of 33 minutes, very beautiful pellets are obtained. A good distribution of the pellets is obtained in accordance with the graph represented in FIG. 2.

The present invention thus has a great many advantages. This is because it makes it possible to provide hard gelatin capsules of the SMEDDS type which are filled by equipment conventional for a multiparticulate solid form.

In addition, the pellets of the invention exhibit the advantage of greatly improving the bioavailability of the active principle.

Finally, the process for the manufacture of these pellets by melt pelletization is fast and simple to employ and can be carried out in conventional equipment of the high-speed mixer type.

1. Pellet for the immediate release of active substance which is intended to be ingested, the said pellet comprising at least one active substance, one binder and one diluent, characterized in that the binder:
comprises a mixture of polyglycolysed glycerides, the tatty acids of which comprise at least 8 carbon atoms,
exhibits a melting point of greater than or equal to 37° C.,
exhibits a hydrophilic-lipophilic balance of greater than or equal to 10,
the said binder being capable of improving the bioavailability of the active substance.

2. Pellet according to claim 1, characterized in that the binder comprises a mixture of saturated polyglycolysed glycerides, the fatty acids of which comprise from 8 to 18 carbon atoms (C₈-C₁₀), exhibits a melting point of 440C and exhibits a hydrophilic-lipophilic balance of 14.

3. Pellet according to either of claims 1 and 2, characterized in that the binder comprises a mixture of polyglycolysed glycerides with C₂₂ fatty acids.

4. Pellet according to one of claims 1 to 3, characterized in that the concentration of binder represents between 10 and 40% by weight of the pellet.

5. Pellet according to one of claims 1 to 4, characterized in that the concentration of binder represents between 15 and 20% by weight of the pellet.

6. Pellet according to one of claims 1 to 5, characterized in that the binder can additionally comprise an additive chosen from the group consisting of polyethylene glycol, esters of glycerol, esters of polyethylene glycol, esters of propylene glycol and esters of polyglycerol with saturated and/or unsaturated fatty acids, alone or as a mixture.

7. Pellet according to one of claims 1 to 6, characterized in that the diluent is provided in the micronized form with a size of between 1 and 300 micrometers.

8. Pellet according to one of claims 1 to 7, characterized in that the active substance is provided in the micronized form with a size of between 1 and 300 micrometers.

9. Process for the manufacture of pellets according to one of claims 1 to 8, characterized in that:
first of all, the active substance, the binder and the diluent are introduced into a mixer equipped with a planetary stirring and heating system,
the constituents are then stirred, while heating, until a temperature close to the melting point of the binder is reached,
the stirring of the mixture obtained is continued until the diluent and the active substance are homogeneously dispersed in the binder,
the stirring and the temperature are maintained in order to make possible the agglomeration, the densification and the spheronization of the particles formed until individual pellets are obtained,
the individual pellets are discharged from the mixer, still with stirring and at a temperature substantially equal to that of the melting point of the binder,
finally, the pellets are recovered and are cooled to a temperature which makes it possible to solidify and to maintain the individual pellets separate from one another.

10. Process according to claim 9, characterized in that the cooling of the pellets is carried out on trays.

11. Process according to claim 9, characterized in that the cooling of the pellets is carried out by means of a fluidized air bed.

12. Process according to one of claims 9 to 11, characterized in that the cooled pellets are dispensed into sachets or hard gelatin capsules.
